(19) United States
(12) Patent Application Publication
(10) Pub. No.: US 2003/0195183 A1
(43) Pub. Date: Oct. 16, 2003

(54) Method for correcting immune system of live body

(76) Inventor: Valerii Khazhumuratovich Zhilov, Moscow (RU)

Correspondence Address: NOTARO AND MICHALOS, 100 DUTCH HILL ROAD, SUITE 110, ORANGEBURG, NY, 10962-2100, US

(21) Appl. No.: 10/348,494

(22) Filed: Jan. 21, 2003

Related U.S. Application Data
(62) Division of application No. 09/896,585, filed on Jun. 29, 2001, now abandoned.

(30) Foreign Application Priority Data
Aug. 2, 2000 (RU) 2000120330

(51) Int. Cl. A61K 31/555; A61K 31/502

(52) U.S. Cl. 514/185; 514/248

(57) ABSTRACT

The present invention relates to medicine and veterinary, namely, to immunology, and to methods aimed at correcting the immune system of a living organism. The essence of the invention is in the immunocorrection of the organism using pharmacologically appropriate amino derivatives of 2,3-dihydrophthalazine-1,4˜dione in their effective doses ranging from 0.2 μg to 1,000 mg.

### CROSS REFERENCE TO RELATED APPLICATION(S)

This is a divisional of application Ser. No. 09/896,585, filed Jun. 29, 2001 which claims priority on Russian Application No. 2000-120330, filed Feb. 8, 2000.

### SUMMARY, BACKGROUND AND DETAILED DESCRIPTION OF THE INVENTION

This invention belongs to a new field of medicine and veterinary—immunology and can be used for preventing and treatment of various diseases associated with immunopathological changes, such as toxicoinfectious, oncologic, allergic and other diseases.

Immunostimulating and conversely, immunodepressive effects are demonstrated by many well-known preparations. For example, tactivine, decaris and dibazole have immunostimulating effects, while mercaptopurine and cyclophosphamide show the immunodepressive effects. However, the majority of well-known preparations exert unidirectional effects on the immune system.

It is well known that sodium salt of 2-amino-1,2,3,4-tetrahydrophthalazine-1,4-dione, dihydrate, which is administered to patients with weak cellular immunity reactions (for example, patients with malignant tumors), causes activation of macrophages, interleukins and other acute phase proteins. This preparation is administered within a dose range of 10 to 1000 mg in the form of injections (for example, 100 mg in 1 ml of water) or per os, for example, 1000 mg in isotonic solution (RF Patent No. 2,113,222, Class A 61 K 31/04,1998).

The invention presents a method of immunocorrection with application of a large group of pharmacologically adequate salts of amino-derivatives of 2,3-dihydrophthalazine-1,4-dione used in effective dose ranging from 0.2 pg to 1000 mg.

The difference of new invention from its prototype is in that it increases the number of immunocorrecting amino phthalazine dione compounds, and considerably widens their field of application both as immunodepressants and immunostimulators in medicine and veterinary, with considerably increased range of effective doses as compared to the prototype.

This invention is using, as immunocorrecting agents the well-known pharmacologically adequate compounds (which have been used previously for other purposes, for example, fungicides (U.S. Pat. No. 2,654,689, Class 514-248, published in 1953)) of the following general formula:

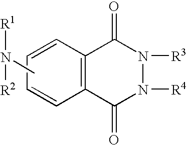

where R₁ R₂, R₃ and R₄ are H-, alkyl-, aryl-, alkylaryl-, atoms of metals or anions.

Such immunologically active compounds of this group include, for example:

lithium, potassium, sodium, calcium, barium, magnesium and silver salts of 5-amino-2,3-dihydrophthalazine-dione

lithium, potassium, sodium, calcium, barium, magnesium and silver salts of 6-amino-2,3-dihydrophthalazine-1,4-dione;

5-amino-2,3-dihydrophthalazine-1,4-dione hydrochloride, sulfate, phosphate, citrate, tartrate, fumarate, oxalate, maleate, acetate, nitrate and hydrobromide;

corresponding salts of 6-amino-2,3-dihydrophthalazine-1,4-dione;

5-methyl amino- and 6-methyl amino-2,3-dihydrophthalazine-1,4-diones and their corresponding, pharmacologically adequate, salts;

5,5-dimethyl amino-, 5,5-diethyl amino-, 5,5-dipropyl amino-, 5,5-dibutyl amino-, 5,5-dipentyl amino-, 6,6-dimethyl amino-, 6,6-diethyl amino-, 6,6-dipropyl amino-, 6,6-dibutyl amino- and 6,6-dipentyl amino-2,3-dihydrophthalazine-1,4-dione and their corresponding, pharmacologically adequate, salts and 5-phenylamino- and 6-phenylamino-2,3-dihydrophthalazine-1, 4-diones and their corresponding, pharmacologically adequate, salts.

The effective dose range of 0.2 μg to 1000 mg was determined experimentally. Selection of specific effective doses within this range for specific cases depends on the nature of disease, body weight and age of patient or animal; this is confirmed below by the data presented in examples and tables. Immunocorrectors may be administered in the form of injections, oral doses or external applications.

Immunogenic and allergenic properties of the advised compounds have been evaluated in accordance with Methodological Recommendations of Ministry of Health of the Russian Federation (R. M. Khaitov, I. S. Gushchin, B. V. Pinegin and A. I. Zebrev. “Experimentalnoye izucheniye immunotropnoy aktivnosti farmakologicheskikh preparatov” (Experimental study of pharmacologic preparations immunotropic activity) Vedemosti farmaklogicheskogo komiteta (Pharmacological Committee Gazette), 1999, No. 1, pp. 31-36).

Mice belonging to various strains were used in this study depending on the applied method of investigation. All mice were supplied by “Stolbovaya” Russian Federation Academy of Medical Sciences (RF AMS) Brooder. Strains CBA and C₅₇BL₆ mice weighing 16 to 18 g were used in the studies of anti-body production and antibody-producing cells.

Effects of the compounds on lymphoid cells proliferation and interleukin-2 induction in vitro were evaluated with the use of Strain BalB/c mice weighing 16 to 18 g. Animals of control and study groups were matched by sex and weight. Each dose of the preparation was tested on 10 animals; as a whole, more than 800 mice have been studied.

Allergenic effects of the preparation were evaluated on female guinea pigs weighing 250 to 300 g supplied by RF AMS Central Brooder of Laboratory Animals. A total of 70 guinea pigs have been studied. Intraperitoneal and intradermal injections of the preparations were used.

Production of antibodies and antibody-producing cells was studied on the model of ram erythrocytes placed in Olswer's solution. The erythrocytes were supplied by M. P. Chumakov Institute of Poliomyelitis and Viral Encephalites Brooder.

The invention can be illustrated by the following examples:

### EXAMPLE 1

### Study of the Effect of Calcium Salt of 5-amino-2,3-dihydrophthalazine-1,4-dione on a Non-Specific Resistivity of the Body

Doses of the preparation ranging from 200 to 0.2 μg were injected intraperitoneally at ten-fold intervals in 0.5 ml of physiologic solution to Strain C₅₇BL₆ mice two hours before their infecting. Mice were infected with S. Typhi in a dose of 1·10⁴ microbial cells; another group of animals was infected with Strain 264 E. coli in a dose of 1·1⁸ microbial cells. Mice infected with the same microbial culture, but receiving no preparation, were used a controls. Lethal outcomes in mice were recorded every day for a period of 10 days.

It was revealed as result of the experiment, that the effects of the preparation on the non-specific resistivity of Strain C₃₇BL₆ strain mice depended on the administered dose and the used model of infection.

Doses of the preparation ranging from 200 to 2 μg caused no effects on the resistivity pf mice infected with E. Coli, and their lifetime was the same as in the controls (See Table 1).

TABLE 1
Evaluation of the effects of calcium salt of 5-amino-
2,3-dihydrophthalazine-1,4-dione on the non-specific
resistivity of mice infected with E. Coli.

| | | Infecting with Strain 264 E. Coli | | | | | | | |
|---|---|---|---|---|---|---|---|---|---|
| | | at a dose of 10⁸ microbial cells | | | | | | | |
| Dose of the | Number | Number of lethal outcomes, | | | | | | | |
| preparation, | of | for days | | | | | | | Mean |
| μg | mice | 1 | 2 | 3 | 4 | 5 | 6 | 7 | lifetime |
| 200 | 10 | 10 | — | — | — | — | — | — | 1.0 |
| 20 | 10 | 10 | — | — | — | — | — | — | 1.0 |
| 2 | 10 | 10 | — | — | — | — | — | — | 1.0 |
| 0.2 | 10 | 4 | — | 6 | — | — | — | — | 2.2 |
| Controls | 10 | 10 | | | | | | | 1.0 |

Thus, the dose of 0.2 μg ensured statistically significant (2.2-fold) increase in lifetime of experimental animals infected with E. Coli.

### EXAMPLE 2

Study of the Effect of Potassium Salt of 5-amino-2,3-dihydrophthalazine-1,4-dione on Phagocytosis In Vivo and In Vitro

Strain C₅₇BL₆ mice received various doses of the preparation ranging from 200 to 2 μg, and 24 hours later they were tested. Intraperitoneal injection of 3 ml of 3% peptone was made, and 2 hours later the animals were killed with chloroform. The mice were autopsied in aseptic conditions. Liquid was aspirated from abdominal cavity with a Pasteur's pipette and transferred to centrifuge test tubes; then centrifuging was carried out for 10 minutes at 1,000 rpm. The sediment was re-suspended in medium 199; number of cells were counted in Goryaev's chamber, and cell concentration was brought to 2·10⁶ neutrophils/ml. Similar volume of Strain 1991 St. aureus was added to cells in a proportion of 1:10 proportion and incubated at 37° C. for 30 minutes. After incubation, smears were prepared on slides, fixed in methanol for 20 minutes and processed with Romanovsky-Giemsa's stain for 30 minutes.

For evaluating the phagocytic activity of macrophages, the animals were tested on the third day after administration of peptone. Further testing was carried out similarly with that of neutrophils. The results were evaluated using a microscope at a magnification of 90×. Phagocytic index and phagocytic number were determined.

Results of the experiment have shown, that the preparation administered in a dose of 200 μg caused no stimulating effect on phagocytic cells, while doses of 2 ug and 20 ug ensured a statistically significant increase in the ability of phagocytic cells to intake microbial cells.

For evaluating the effect of the preparation on phagocytosis in vitro, blood sample was collected from donor's cubital vein in a test tube containing heparin at a dose of 10 units per ml of blood. 0.8 ml of 3% gelatin prepared based on Medium 199 were added to 2 ml of blood. The test tubes were incubated in a thermostat at 37° C. for 20 minutes. Then the supernatant containing cells was aspirated to a centrifuge tube. Cells were twice washed with Medium 199 with centrifuging at 1,000 rpm. 1 ml of Medium 199 was added to the sediment and neutrophils were counted in Goryaev's chamber. Then, cellular suspension containing 2·10⁶ neutrophils in 1 ml was prepared based on Medium 199. Simultaneously, suspension of St. aureus in a concentration of 20·10⁶/ml was prepared. After preliminary opsonization with an equal volume of pooled human plasma in a thermostat at 37° C. for 20 minutes, the Staphylococcus suspension was centrifuged and washed with Medium 199. The preparation in 200, 20 and 2 μg/ml concentrations was added to mixture of equal volumes of neutrophils and staphylococci; Medium 199 was added to control test tubes. After incubation in a thermostat at 37° C. for 30 minutes, the test tubes where centrifuged, and preparations were prepared in accordance with the procedure described above. Phagocytic index and phagocytic number for study group were compared with the same for control group.

The obtained results are presented in Table 2:

TABLE 2
Effects of various doses of potassium salt of 5-amino-
2,3-dihydrophthalazine-1,4-dione on neutrophil
phagocytosis in vitro.

| | Study Group | | Control Group | |
|---|---|---|---|---|
| | Phagocytic | Phagocytic | Phagocytic | Phagocytic |
| Dose, μg | number | Index | Number | Index |
| 1 | 2 | 3 | 4 | 5 |
| 200 | 78 | 5.6 | 77 | 6.1 |
| | 85 | 4.7 | 86 | 5.1 |
| | 84 | 9.1 | 85 | 8.7 |
| | 71 | 6.4 | 72 | 7.2 |
| | 67 | 7.4 | 68 | 8.1 |
| Mean | 77 ± 0.26 | 6.0 ± 0.04 | 77.6 ± 0 | 7.0 ± 0.04 |
| 20 | 80 | 3.9 | 76 | 4.2 |
| | 96 | 2.8 | 66 | 3.5 |
| | 88 | 4.6 | 82 | 6.6 |
| | 94 | 6.8 | 82 | 5.6 |
| | 84 | 13.2 | 83 | 5.0 |
| | 96 | 9.1 | 92 | 10.0 |
| | 86 | 16.3 | 74 | 5.5 |
| | 84 | 5.2 | 62 | 4.4 |
| Mean | 89 ± 1.25 | 7.8 ± 0.04 | 77 ± 0.35 | 5.6 ± 0.1 |
| | 88 | 6.4 | 87 | 6.2 |
| | 92 | 4.5 | 80 | 7.3 |
| | 78 | 7.4 | 30 | 5.8 |
| | 84 | 6.4 | 80 | 5.5 |
| | 92 | 5.6 | 80 | 9.1 |
| Mean | 86 ± 0.32 | 6.0 ± 0.16 | 71.4 ± 0 | 6.8 ± 0.2 |

The obtained data show, that the injected doses of the preparation ranging from 200 to 2 μg caused stimulating effects on phagocytic activity of macrophages in mice, while the dose of 200 μg caused no effect on phagocytic activity in vivo.

It has been found in the in vitro experiments that the studied preparation used at the doses of 20 and 2 μg caused statistically significant increase in the ability of neutrophils population to intake microbial cells and demonstrated practically no effect on their digestive activity.

### EXAMPLE 3

### Study of the Effect of Sodium Salt of 5-amino-2,3-dihydrophthalazine-1,4-dione on the Humoral Immune Response

Mice were immunized intraperitoneally with ram erythrocytes washed with physiologic solution; the erythrocytes were injected at a dose of 5·10⁶ cells. Other groups of animals received ram erythrocytes and doses of the preparation ranging from 200 to 0.2 μg at ten-fold intervals. When productive phase of humoral immunity response was studied, the preparation was administered to mice on the fifth day after their immunization with erythrocytes. Blood samples of the mice were collected on the 7^{th}, 14^{th} and 21^{st} day after the immunization. Antibodies were determined using a reaction of hemagglutination. Two-fold dilutions of mice blood sera were studied in 96-well plates for immunological reactions with U-shaped bottoms in 25-μl volumes. 25 μl of physiologic solution were added to control well. 25 ul of 1% solution of ram erythrocytes were added to each well. The plates were incubated in a thermostat at 37° C. for 2 hours. The last dilution of the studied serum demonstrating positive result was considered as a titer. The control well had to be negative.

When the effect of the preparation on inductive phase of immune response was studied, the preparation was administered to mice simultaneously with ram erythrocytes. Blood was collected from mice on the 7^{th}, 14^{th} and 21^{st} day after immunization. Antibodies were determined using a reaction of hemagglutination as described above.

Results obtained after simultaneous administration of ram erythrocytes (RE) and the preparation are shown in Tables 3 and 4. Strain C₅₇BL₆ animals of low reaction showed a tendency to increase in titers of hemagglutinins on the 21^{st} day after the administration of 0.2 or 20 μg of the preparation to each mouse. Study of the effect of the preparation on productive phase of immune response of mice revealed a suppressive effect of all doses of the preparation on Strain CBA mice of high reaction, as well as a tendency to increase in the level of hemagglutinins on the 21^{st} day after administration of the preparation to low reaction Strain C₅₇BL₆ mice:

TABLE 3
Titers of hemagglutinins after simultaneous
administration of sodium salt of 5-amino-2,3-
dihydrophthalazine-1, 4-dione and RE.

| Dose of the | | | | | | |
|---|---|---|---|---|---|---|
| preparation | Strain CBA mice | | | Strain C₅₇BL₆ mice | | |
| μg | 7 days | 14 days | 21 days | 7 days | 14 days | 21 days |
| 0.2 | 112 ± 106 | 168 ± 109 | 15 ± 13 | 59 ± 58 | 50 ± 48 | 266 ± 200 |
| 2 | 28 ± 26 | 154 ± 97 | 10 ± 8 | 154 ± 57 | 256 ± 0 | 24 ± 20 |
| 20 | 96 ± 37 | 256 ± 0 | 83 ± 70 | 91 ± 64 | 512 ± 0 | 560 ± 160 |
| 200 | 35 ± 33 | 75 ± 59 | 46 ± 32 | 512 ± 0 | 44 ± 42 | 160 ± 138 |
| RE | 112 ± 106 | 512 ± 0 | 24 ± 12 | 154 ± 57 | 256 ± 0 | 27 ± 15 |
| Controls | 0 | 4 ± 0 | 0 | 0 | 4 ± 0 | 0 |

TABLE 4
Titers of hemagglutinins revealed in mice receiving
the preparation five days after immunization with RE.

| Dose of the | | | | | | |
|---|---|---|---|---|---|---|
| preparation | Strain CBA mice | | | Strain C₅₇BL₆ mice | | |
| μg | 7 days | 14 days | 21 days | 7 days | 14 days | 21 days |
| 0.2 | 0 | 9 ± 7 | 0 | 0 | 2 ± 0 | 35 ± 10 |
| 2 | 10 ± 8 | 173 ± 110 | 22 ± 10 | 13 ± 11 | 54 ± 50 | 226 ± 201 |
| 20 | 0 | 236 ± 0 | 23 ± 10 | 26 ± 19 | 256 ± 0 | 304 ± 105 |
| 200 | 28 ± 13 | 158 ± 120 | 47 ± 30 | 0 | 96 ± 42 | 120 ± 95 |
| RE | 111 ± 106 | 512 ± 0 | 24 ± 12 | 154 ± 57 | 256 ± 0 | 27 ± 15 |
| Controls | 0 | 4 ± 0 | 0 | 0 | 4 ± 0 | 0 |

Thus, the ability of sodium salt of 5-amino-2,3-dihydrophthalazine-1,4-dione to change the antibody production depending on its dose and initial immunologic reactivity of the body has been revealed. Administration of the preparation during productive phase, that is, on the 5^{th} day after injection of antigens (ram erythrocytes), resulted in inhibition, with wide range of doses, of production of antibodies on the 7^{th} and 14^{th} days after immunization of Strain CBA mice, which are, genetically, highly sensitive to ram erythrocytes. In low-sensitive Strain C₅₇BL₆ the preparation caused a significant increase in the titers of hemagglutinins in low-sensitive Strain C₅₇BL₆ mice on the 21^{th} day of the experiment.

### EXAMPLE 4

### Study of the Effect of Sodium Salt of 6-amino-2,3-dihydrophthalazine-1,4-dione on the Cellular Immune Response

To evaluate the effect of the preparation on cellular immunity reaction, delayed hypersensitivity reaction (DHR) was used. The mice were sensitized by subcutaneous injection of 1·10⁷ ram erythrocytes contained in a volume of 20 μl. Doses of the preparation ranging from 0.2 to 2,000 μg were injected at ten-fold intervals simultaneously with sensitizing and shocking dose of the antigen. Shocking dose of ram erythrocytes (1·10⁸) was injected under aponeurotic plate of left hind paw on the 5^{th} day after sensitization. Injection of 20 μl of physiologic solution in the right paw served as control. Intensity of inflammatory reaction was recorded 24 hours after the administration of shocking dose of the antigen. For this purpose, the mice were killed and, immediately after this, both paws were amputated at the level of ankle joint and weighed on torsion balance. Reaction index (RI) was determined as the difference of masses of study (S) and control (C) paws using the following formula:

$$RI = \frac{S - C}{C} \times 100 \%$$

This study evaluating the effects of the preparation on cellular immune response based on DHR has revealed a tendency to increase in reaction index for mice of both strains as the dose of the preparation decreased (see Table 5). It should be noted, that the increase in reaction index accompanying the decrease in the dose of preparation was especially prominent in low-reaction Strain C₅₇BL₆ mice. Administration of the preparation at a dose of 200 μg to high-reaction Strain CBA mice resulted in inhibition of DHR.

TABLE 5
Effect of sodium salt of 6-amino-2,3-
dihydrophthalazine-1,4-dione on delayed
hypersensitivity reaction (DHR) in mice

| | |
|---|---|
| Dose of the | |
| preparation, | Reaction index |

| μg | Strain CBA mice | Strain C₅₇BL₆ mice |
|---|---|---|
| 2 | 11 ± 8 | 19 ± 9 |
| 20 | 10 ± 5 | 16 ± 9 |
| 200 | 2 ± 1.5 | 15 ± 6 |
| Control | 6 ± 3.5 | 12 ± 7 |

Thus, administration of 200 μg of the preparation to Strain CBA mice suppressed the development of DHR. All other doses of the preparation (20 to 2 μg) showed no effect on the development of DHR in mice of both oppositely reacting strains.

### EXAMPLE 5

### Study of the Effect of 5-amino-2,3-dihydrophthalazine-1,4-dione Hydrochloride on Lymphoid Cells Proliferation

Spleens of the animals were used for carrying out the immunologic reactions. The organs were cut and homogenized in a glass homogenizer. The homogenate was filtered through stainless steel filters with pores measuring 50 to 100 μm in diameter and then washed three times in a medium designed for centrifuging (CM) consisting of Medium 199 with 5% embryonic calf serum (produced by R F AMS N. F. Gamaleya NIIEM), 1 mM of HEPES buffer solution and 50 μg/ml gentamicin. The suspensions were centrifuged in a K23 centrifuge at 1,500 rpm at 4° C. for 10 minutes.

The suspensions were diluted 100-fold with 3% acetic acid contrasted with methylene blue, and cells were counted in Goryaev's chamber. Cell viability was evaluated with aid of 0.1% trypan blue in physiologic solution.

Results fo the study of mitogenic effects of the preparation, as well as the effects of the preparation on proliferation caused by T-cell (KonA) and B-cell (LPS) mitogens, are presented in Table 6.

TABLE 6
Effect of 5-amino-2,3-dihydrophthalazine-1,4-dione
hydrochloride on lymphoid cells proliferation.

| Concentration of | Mitogen added to spleen cell culture | | |
|---|---|---|---|
| the preparation | — | ConA | LPS |
| — | 3534 ± 563 | 17896 ± 2080 | 18874 ± 355 |
| 50 μg/ml | 2576 ± 237 | 11860 ± 1566 | 13232 ± 928 |
| 500 μg/ml | 1763 ± 94 | 1323 ± 192 | 3870 ± 308 |
| 2.5 μg/ml | 249 ± 93 | 195 ± 27 | 251 ± 44 |
| 12.5 μg/ml | 369 ± 56 | 178 ± 28 | 256 ± 40 |

As the presented data show, the preparation has no mitogenic properties within the studied dose range (50 μg/ml to 12.5 mg/ml). However, this preparation, when used in higher concentrations, inhibited both the spontaneous proliferation of spleen cells and the proliferation induced by nonspecific mitogens (ConA and LPS).

LPS=Lipopolysaccharide

ConA=Concanavaline A

### EXAMPLE 6

### Study of the Effect of Sodium Salt of 5-methylamino-2,3-dihydrophthalazine-1,4-dione on Functional Activity of Natural Killers

Labeled cells of mice of myeloblastoid Strain K-562, as well as mononuclear cells of 10 healthy blood donors and 10 patients with various diseases with low and high levels of cytotoxicity, were used for the study of effects of various concentrations of the preparation on cytotoxic activity of natural killers (CD3⁻, CD16⁺ and CD56⁺).

The used Strain K-562 cells to effector cells ratio was 1:25 (100 μl of labeled cells and 100 μl of mononuclear cells). The studies were carried out using 96-well plates; incubation for 16 to 24 hours in a CO₂-incubator at 37° C. was carried out. Then, the contents of the wells was transferred to filters, washed, dried and placed in solution with scintillation liquid with consecutive determination of index using a β-counter. Cytotoxic index (CI) was calculated using the following formula:

$$CI = 1 - \frac{( A - B )}{( C - B )} \times 100 ,$$

where

A=the target cells radioactivity in the presence of effector cells;

B=radioactivity after cells treatment with tribon X-100 (maximum value);

C=radioactivity of target cells in the absence of effector cells.

The obtained results are shown in Tables 7 and 8 below.

TABLE 7
Effects of various concentrations of sodium salt of 5-
methylamino-2,3-dihydrophthalazlne-1,4-dione on
cytotoxic index depending on control level of CI.

| Preparation concentration, μg/ml | CI, % |
|---|---|
| Control | 41.0 ± 3.0 |
| 2.5 | 41.9 ± 2.8 |
| 5.0 | 38.8 ± 3.1 |
| 10.0 | 40.8 ± 3.0 |
| 25.0 | 41.4 ± 3.0 |
| 50.0 | 39.1 ± 4.0 |
| 75.0 | 37.7 ± 4.1 |
| 150.0 | 41.0 ± 3.5 |
| 300.0 | 39.0 ± 3.1 |

The obtained results have shown, that various concentrations of the preparation (within 2.5 to 300 μg/ml range) did not cause significant modification of natural killers cytotoxicity when normal level of CI was present.

TABLE 8
Effects of low concentrations of sodium salt of
5-methylamino-2,3-dihydrophthalazine-1,4-dione on
cytotoxic index depending on control level of CI

| Concentration of | | | |
|---|---|---|---|
| the preparation, | Cytotoxic index level, % (M) | | |
| μg/ml | Medium | High | Low |
| Control | 41.0 ± 0.8 | 64 ± 0.8 | 20.7 ± 1.0 |
| 2.5 | 41.9 ± 0.9 | 57 ± 0.7 | 34 ± 1.3 |
| 5.0 | 38.S ± 0.34 | 54 ± 0.8 | 30 ± 1.2 |
| Number of | 10 | 10 | 10 |
| observations | | | |

It follows from the data presented in Table 8, that the preparation, when used in 2.5 and 5.0 μg/ml doses in conditions of high initial level of CI, caused statistically significant inhibition of cytotoxicity, while in conditions of low initial level of CI it caused statistically significant stimulation of functional activity of natural killers, that is, it demonstrated the ability to modulate the cytotoxicity depending on its initial level.

### EXAMPLE 7

### Study of Anaphylactogenic Activity of Sodium Salt of 5-amino-2,3-dihydrophthalazine-1,4-dione

The preparation under study was injected to three groups of guinea pigs in accordance with the following schedule: first 0.1-ml injection of various doses (1 μg, 10 μg and 100 μg) was made subcutaneously; second 0.1-ml injection of the preparation was made on the next day intramuscularly in the thigh; and the third 0.1-ml injection was made one day later. On day 21, the shocking injection of main pharmacological effect with single application was made. The obtained results are shown in Table 9.

TABLE 9
Expression of anaphylactic shock

| Sensitization | | | | | | | | |
|---|---|---|---|---|---|---|---|---|
| with the | Shocking | Number | Expression of | | | | | |
| preparation, | Injection | of | anaphylactic shock | | | | | Weigl's |
| μg | μg | animals | − | + | ++ | +++ | ++++ | index |
| 100 | 100 | 10 | 5 | 1 | 1 | — | — | 0.3 |
| 10 | 100 | 10 | 7 | 2 | — | — | — | 0.2 |
| 1.0 | 100 | 10 | 10 | — | — | — | — | |

Thus, the expression of anaphylactic activity of the studied preparation was very weak; it was demonstrated only for high doses of the preparation.

Apart from the studies on animals described above, clinical studies of immunocorrection activity of amino derivatives of 2,3-dihydrophthalazine-1,4-dione represented by sodium salt of 5-amino-2,3-dihydrophthalazine-1,4-dione in the treatment of various diseases on human, in particular, anti-inflammatory immunocorrecting activity in the treatment of ulcers, were carried out.

Examples 8 and 9 illustrate the results of clinical studies.

### EXAMPLE 8

Patient K aged 35 years had a chronic recurrent ulcer on the antero-inferior wall of duodenal bulb measuring about 1.5 cm in diameter and about 0.5 cm in depth; another ulcer measuring 0.3 cm in diameter was present on the opposite wall (“kissing ulcer”). Concomitant catarrhal bulbitis was present. The patient partly lost her working capacity due to this disease.

Treatment: Periulceral injection of 100 mg of sodium salt of 5-amino-2,3-dihydrophthalazine-1,4-dione was made. One more injection of the preparation was made on the 7^{th} day. The patient also received the preparation in Almagel suspension and, since the 7^{th} day, in Maalox (in view of constipations). She was simultaneously treated with omeprazol in accordance with usual regimen and, since the 7^{th} day, De-nol with trichopol.

Significant weakening of pain was noted on the 2^{nd} day of treatment; the pain completely disappeared on the 7^{th} day. By the 7^{th} day, decrease in ulcer dimensions by ⅓, clearing of the bottom of ulcerous defect, epithelialization of the upper wall of ulcer and decrease in the manifestations of bulbitis were noted. By the 15^{th} day of treatment, the dimensions of ulcerous defect decreased by 2/3. The ulcer completely scarred by the 21^{st} day. The patient received a total of 1, 000 mg of the preparation in the course of treatment.

### EXAMPLE 9

### Study of the Efficacy of Potassium Salt of 5-amino-2, 3-dihydro-phthalazine-1,4-dione in the Treatment of Acute Intestinal Infections (AII)

Efficacy of the preparation in decreasing the gravity of manifestations of various forms of AII was evaluated in accordance with the used program of study with aid of a three-point scale. In 19 of 20 patients, the intoxication syndrome resolved within 1 to 3 days of the disease (“complete decrease” in the gravity of disease), and one patient demonstrated “insignificant decrease” in the course of the first days and “complete decrease” by the 5^{lh} day of treatment.

After the study was finished, general evaluation of clinical efficacy and safety of the preparation based on a 4-point scale, depending on nosologic forms, was carried out. Results of this evaulation are presented in Table 10.

TABLE 10
Efficacy of potassium salt of 5-amino-2,3-
dihydrophthalazine-1,4-dione in the treatment of
various forms of AII.

| | Total | | | | |
|---|---|---|---|---|---|
| | number | Efficacy of the preparation | | | |
| Form of | of | | | | No |
| disease | patients | Excellent | Good | Satisfactory | effect |
| Acute | 9 | 1 | 7 | 1 | — |
| dysentery | | | | | |
| Salmonellosis | 3 | 1 | 2 | — | — |
| Alimentary | 4 | 1 | 3 | — | — |
| toxoinfection | | | | | |
| Acute | 4 | 2 | 1 | 1 | — |
| gastroenteritis | | | | | |
| Total: | 20 | 5 | 13 | 2 | |

Thus, the preparation has demonstrated high efficacy in the treatment of all 20 patients with various forms of AII; the preparation showed “excellent” or “good” effect in 90% of patients (18 patients).

It follows from the data presented above, that the studies of immunotropic activity of amino derivatives of 2,3-dihydrophthalazine-1,4-dione have revealed their dose-dependent immunocorrecting activity within the 0.2 pg to 1,000 mg dose range.

What is claimed is:

1. Pharmacological preparation for correcting the immune system of a living organism using aminodihydrophthalazine compounds, characterized in that the immunocorrecting agent is an amino derivative of 2,3-dihydrophthalazine-1,4-dione or a pharmacologically adequate salt thereof.

2. Pharmacological preparation according to claim 1, characterized in that the immunocorrecting agent is preferably a compound according to the following general formula: 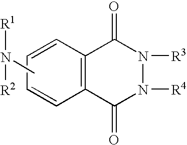
where, R¹, R², R³ and R⁴ are H-, alkyl-, aryl-, alfcylaryl-, atoms of metal or anion and/or a derivative and/or a salt thereof.

3. Pharmacological preparation according to claim 1, wherein the immunocorrecting agent is a lithium, potassium, sodium or calcium, barium, magnesium and/or a silver salt of 5-amino-2,3-dihydrophthalazine-1,4-dione.

4. Pharmacological preparation according to claim 1, wherein the immunocorrecting agent is a lithium, potassium, sodium, calcium, barium, magnesium and/or a silver salt of 6-amino-2,3-dihydrophthaiazine-1,4-dione.

5. Pharmacological preparation according to claim 1, wherein the immunocorrecting agent is a 5-amino-2,3-dihydrophthalazine-1,4-dionehydrochloride-sulfate, -phosphate, -citrate, -tartrate, -furoarate, -oxalate, -realeate, -acetate/-nitrate and/or -hydrobromide.

6. Pharmacological preparation according to claim 1, wherein the immunocorrecting agent corresponds to a salt of 6-amino-2,3-dihydrophthalazine-1,4-dione.

7. Pharmacological preparation according to claim 1, wherein the immunocorrecting agent corresponds to 5-methyl amino- and/or 6-methyl amino-2,3-dihydrophthalazine-1,4-diona and/or corresponding/pharmacologically adequate salts.

8. Pharmacological preparation according to claim 1, wherein the immunocorrecting agent is 5/5-dimethyl amino-, 5,5-diethyl amino-, 5,5-dipropyl amino-, 5,5-dibutyl amino-/5,5,-dipentyl amino-, 6/6-dimethyl amino-, 6,S-diethyl amino-, 6,6-dipropyl amino-, 6,6-dibutyl amino- and/or 6,6-dipentyl amino-2,3-dihydrophthaline-1,4-dione and/or corresponding, pharmacologically adequate salts.

9. Pharmacological preparation according to claim 1, wherein the immunocorrecting agent is 5-phenylaraino- and/or 6˜phenylamino-2,3-dihydrophthalazine-1,4-dione and/or corresponding, pharmacologically adequate salts.

10. Use of the pharmacological preparation according to claim 1 for correcting the immune system of live bodies with the aid of an immunocorrecting agent as defined in claim 1, which is distinguished by the use as immunocorrectors of pharmacologically adequate amino derivatives of 2,3-dihydrophthalazine-1,4-dione and/or a salt thereof in effective dose ranging from 0.2 μg to 1000 mg.

11. Use of the pharmacological preparation according to claim 1, using as immunocorrectors pharmacologically adequate metallic salt of amino derivatives of 2-3-dihydrophthalazine-1,4-dione.

12. Pharmacological preparation according to claim 10, using as immunocorrecting agent a derivative as claimed.

13. A method for correcting the immune system of living organism with the help of amino dihydrophthalazine compounds, characterized by the use an iromunocorrector of pharmacologically adequate amino derivatives of 2,3-dihydrophthala2ine-1,4-dione in effective doses ranging from 0.2 μg to 1000 mg.

14. A method, in accordance with Example 1, characterized by preferential use, as immunocorrectors, of pharmacologically adequate metallic salts of amino derivatives of 2,3-dihydrophthalazine-1,4-dione.
